(19) United States
(12) Patent Application Publication
(10) Pub. No.: US 2024/0147930 A1
(43) Pub. Date: May 9, 2024

(54) HYBRID BITTER GOURD 'E97B.00040'

(71) Applicant: Enza Zaden Beheer B.V., Enkhuizen (NL)

(72) Inventors: Josephus Van Balen, Bovenkarspel (NL); Uwiragiye Ambroise, Hoorn (NL)

(21) Appl. No.: 18/386,360

(22) Filed: Nov. 2, 2023

Related U.S. Application Data
(60) Provisional application No. 63/423,339, filed on Nov. 7, 2022.

(51) Int. Cl. A01H 6/34; A01H 5/08

(52) U.S. Cl. CPC A01H 6/348; A01H 5/08

(57) ABSTRACT

A seed of hybrid bitter gourd designated as ‘E97B.00040’ is disclosed. The present disclosure includes seeds and/or plants of hybrid bitter gourd ‘E97B.00040’, methods for producing a hybrid plant, and methods for producing other bitter gourd lines, cultivars or hybrids derived from the hybrid bitter gourd ‘E97B.00040’.

### CROSS-REFERENCE TO RELATED APPLICATIONS

This application claims the benefit of priority to U.S. Provisional Application No. 63/423,339, filed on Nov. 7, 2022, the entire content of which is hereby incorporated by reference.

### FIELD

The present disclosure relates to the field of plants and plant breeding. In particular, this disclosure includes a new and distinctive bitter gourd, Momordica charantia L., hybrid designated ‘E97B.00040’.

### BACKGROUND

Bitter gourd (Momordica charantia L.) is a member of the family Cucurbitaceae. The Cucurbitaceae is a family of about 90 genera and 700 to 760 species, mostly of the tropics. The family includes melons, pumpkins, squashes, gourds, watermelon, loofah and many weeds. The genus Momordica includes about 60 species of annual and perennial vines native to tropical and subtropical Africa, Asia, and Australia, of which bitter gourd is the most commercially important. Like typical Cucurbitaceae, bitter gourd has unisexual flowers that usually require pollen vectors to set fruit. Bitter gourd plants are monoecious, as are other members of Momordica section Momordica.

Bitter gourd is a frost-intolerant annual or perennial crop grown primarily for its fresh fruit, which is treated as a culinary vegetable. Bitter gourd cultivation is similar to cucumber, with vines grown on support structures like trellises. The slender green vines bear simple, unbranched tendrils, petiolate leaves with three to seven lobes, and pendent fruit. Bitter gourd fruit have an irregular surface texture characterized by raised warts and longitudinal ridges. The fruit have a large central cavity containing inedible pith and seeds. Bitter gourds are harvested before full maturity, when the exterior skin color is still white to dark green (depending on variety). At maturity, bitter gourd skin and flesh turns yellow to orange, softens, and splits longitudinally to disperse textured, brownish seeds surrounded by fleshy red arils. Young bitter gourd shoots and leaves are also occasionally eaten as greens, and share the characteristic bitter flavor of the fruit.

Bitter gourds are valued in Asian, African, and Caribbean cuisines for their bitter flavor, which can range from mild to intense depending on variety and cooking method. Bitter gourd fruit and plants, including small semi-feral varieties, also have many ethnobotanical uses due to their phytochemical profile, including purported anti-diabetic properties. While cultivated varieties of bitter gourd have lower levels of some bioactive compounds, the fruit is a good source of vitamin C, folate, zinc, and potassium.

### SUMMARY

Bitter gourd is an important and valuable field crop. Thus, there is a continued need for new hybrid bitter gourds that are stable, high yielding, and/or agronomically sound. There is a need for improved bitter gourds with desirable production characteristics including parthenocarpy, allowing for production without pollen vectors. Further, there is a need for improved characteristics related to specific cultivation methods, such as suitability for greenhouse production. Therefore, there is a need for bitter gourd varieties adapted for particular cultivation environments. In order to meet these needs, the present disclosure is directed to, among other things, improved hybrid bitter gourds.

In one aspect, the present disclosure is directed to a hybrid bitter gourd, Momordica charantia, seed designated as ‘E97B.00040’ (or ‘Sulasih’) having NCIMB Accession Number X1. In some embodiments, the present disclosure is directed to a Momordica charantia bitter gourd plant and/or parts isolated therefrom produced by growing ‘E97B.00040’ bitter gourd seed (which plants and parts can be referred to, e.g., as ‘E97B.00040‘ plants and’E97B.00040’ parts, respectively). In another embodiment of this aspect, the present disclosure is directed to a Momordica charantia plant and/or parts isolated therefrom having all, or essentially all, of the physiological and morphological characteristics of a Momordica charantia plant produced by growing ‘E97B.00040’ bitter gourd seed having NCIMB Accession Number X1.

Bitter gourd plant parts include bitter gourd leaves, ovules, pollen, seeds, bitter gourd fruits, parts of bitter gourd fruits, flowers, cells, and the like. In another embodiment, the present disclosure is further directed to bitter gourd leaves, ovules, pollen, bitter gourd fruits, and/or cells isolated from ‘E97B.00040’ bitter gourd plants. In certain embodiments, the present disclosure is further directed to pollen or ovules isolated from ‘E97B.00040’ bitter gourd plants. In another embodiment, the present disclosure is further directed to protoplasts produced from ‘E97B.00040’ bitter gourd plants. In another embodiment, the present disclosure is further directed to tissue or cell culture of ‘E97B.00040’ bitter gourd plants, and/or to bitter gourd plants regenerated from the tissue or cell culture, where the plants regenerated from the tissue or cell culture has all, or essentially all, of the morphological and physiological characteristics of ‘E97B.00040’ bitter gourd. In certain embodiments, tissue culture of ‘E97B.00040’ bitter gourd plants is produced from a plant part selected from root, root tip, meristematic cell, stem, hypocotyl, petiole, cotyledon, leaf, flower, anther, pollen, pistil, and/or fruit.

In another aspect, the present disclosure is directed to a method of making hybrid bitter gourd ‘E97B.00040’, said method including selecting seeds from the cross of one ‘E97B.00040’ plant with another ‘E97B.00040’ plant, a sample of ‘E97B.00040’ bitter gourd seed having been deposited under NCIMB Accession Number X1.

In a further aspect, the present disclosure is directed to a method of producing a seed of a ‘E97B.00040’-derived bitter gourd plant, including: crossing a hybrid bitter gourd designated as ‘E97B.00040’, representative sample of seed having been deposited under NCIMB Accession Number X1, with itself or a second bitter gourd plant; whereby seed of a ‘E97B.00040’-derived bitter gourd plant forms. In another embodiment of this aspect, the method further includes (b) crossing a plant grown from ‘E97B.00040’-derived bitter gourd seed with itself or a second bitter gourd plant to yield additional ‘E97B.00040’-derived bitter gourd seed; (c) growing the additional ‘E97B.00040’-derived bitter gourd seed of step (b) to yield additional ‘E97B.00040’-derived bitter gourd plants; and (d) repeating the crossing and growing of steps (b) and (c) for at least one additional generation (e.g., for an additional 3-10 generations) to generate further ‘E97B.00040’-derived bitter gourd plants.

In yet another aspect, the present disclosure is directed to a method of vegetatively propagating a plant of hybrid bitter gourd variety ‘E97B.00040’, the method including the steps of: (a) collecting tissue capable of being propagated from a plant of hybrid bitter gourd variety ‘E97B.00040’, representative seed of said hybrid bitter gourd variety having been deposited under NCIMB Accession Number X1; and (b) producing a rooted plant from said tissue. In another embodiment, the present disclosure is further directed to bitter gourd plants, plant parts and/or seeds produced by the bitter gourd plants where the bitter gourd plants are produced by any of the preceding methods of the disclosure.

According to the disclosure, there is provided a hybrid bitter gourd plant designated ‘E97B.00040’. This disclosure thus relates to the seeds of hybrid bitter gourd ‘E97B.00040’ and to the plants of bitter gourd ‘E97B.00040’. This disclosure also relates to methods for producing other bitter gourd cultivars or hybrids derived from hybrid bitter gourd ‘E97B.00040’ and/or to bitter gourd cultivars and hybrids derived by the use of those methods.

In another embodiment, the present disclosure is directed to single gene converted plants of hybrid bitter gourd ‘E97B.00040’. The single transferred gene may preferably be a dominant or recessive allele. Preferably, the single transferred gene will confer important traits including, among other things, herbicide resistance, insect resistance, resistance for bacterial, fungal, or viral disease, improved harvest characteristics, enhanced nutritional quality, and/or improved agronomic quality. The single gene may be a naturally occurring bitter gourd gene or a transgene introduced through genetic engineering techniques.

In another embodiment, the present disclosure is directed to methods for developing bitter gourd plants in a bitter gourd plant breeding program using plant breeding techniques including recurrent selection, backcrossing, pedigree breeding, restriction fragment length polymorphism enhanced selection, genetic marker enhanced selection, and/or transformation. Marker loci such as restriction fragment polymorphisms or random amplified DNA have been published for many years and may be used for selection (See, Pierce et al., HortScience (1990) 25:605-615; Wehner, T., Cucurbit Genetics Cooperative Report, (1997) 20: 66-88; and Kennard et al., Theoretical Applied Genetics (1994) 89:217-224). Seeds, bitter gourd plants, and parts thereof produced by such breeding methods are also part of the disclosure.

In additional embodiments, the present disclosure is directed to bitter gourd seeds resulting from methods of making a bitter gourd variety of the present disclosure. In additional embodiments, the present disclosure is directed to bitter gourd plants, and/or parts thereof, obtained from growing the seeds of the present disclosure. In additional embodiments, the present disclosure is directed to bitter gourd plants, and/or parts thereof, having all, or essentially all, of the physiological and morphological characteristics of the bitter gourd plants of the present disclosure. In additional embodiments, the present disclosure is directed to bitter gourd tissue or cell culture, obtained from the plants of the present disclosure. In further embodiments, the tissue or cell culture of the present disclosure is produced from a plant part selected from the group consisting of root, root tip, meristematic cell, stem, hypocotyl, petiole, cotyledon, leaf, flower, anther, pollen, pistil, and/or fruit.

In addition to the exemplary aspects and embodiments described above, further aspects and embodiments will become apparent by reference by study of the following descriptions.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows whole fruits of hybrid bitter gourd ‘E97B.00040’ harvested at market maturity.

FIG. 2 shows a whole fruit (left) and a longitudinal section of fruit (right) of hybrid bitter gourd ‘E97B.00040’ harvested at market maturity.

FIG. 3 shows a flowering plant of hybrid bitter gourd ‘E97B.00040’.

FIG. 4 shows a comparison between fruits of ‘Bilai’ variety (left) and fruits of hybrid bitter gourd ‘E97B.00040’ (right).

### DETAILED DESCRIPTION

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The selected germplasm is crossed in order to recombine the desired traits and through selection varieties or parent lines are developed. The goal is to combine in a single variety or hybrid an improved combination of desirable traits from the parental germplasm. These important traits may include, among other things, higher yield, field performance, fruit and agronomic quality such as fruit shape and length, resistance to diseases and insects, and tolerance to drought and heat.

Choice of breeding or selection methods can depend on the mode of plant reproduction, the heritability of the trait(s) being improved, and the type of cultivar used commercially (e.g., an F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives, and can include gain from selection per year based on comparisons to an appropriate standard, overall value of the advanced breeding lines, and number of successful cultivars produced per unit of input (e.g., per year, per dollar expended, etc.).

Promising advanced breeding lines can be thoroughly tested and compared to appropriate standards in environments representative of the commercial target area(s) for at least three years. The best lines can then be candidates for new commercial cultivars. Those still deficient in a few traits may be used as parents to produce new populations for further selection. These processes, which lead to the final step of marketing and distribution, may take from ten to twenty years from the time the first cross or selection is made.

One goal of bitter gourd plant breeding is to develop new, unique, and genetically superior bitter gourd cultivars and hybrids. A breeder can initially select and cross two or more parental lines, followed by repeated selfing and selection, producing many new genetic combinations. A plant breeder can then select which germplasms to advance to the next generation. These germplasms may then be grown under different geographical, climatic, and soil conditions, and further selections can be made during, and at the end of, the growing season.

The development of commercial bitter gourd cultivars thus requires the development and/or selection of bitter gourd parental lines, the crossing of these lines, and the evaluation of the crosses. Pedigree breeding and recurrent selection breeding methods may be used to develop cultivars from breeding populations. Breeding programs can be used to combine desirable traits from two or more varieties or various broad-based sources into breeding pools from which lines are developed by selfing and selection of desired phenotypes. The new lines are crossed with other lines and the hybrids from these crosses are evaluated to determine which have commercial potential.

Pedigree breeding is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding may be been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques known in the art that are available for the analysis, comparison and characterization of plant genotype. Such techniques include, without limitation, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), High Resolution Melt (HRM), Genotyping by Sequencing (GbS), and/or Next-generation Sequencing (NGS).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding may also be used to introduce new traits into bitter gourd varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Additional non-limiting examples of breeding methods that may be used include, without limitation, those found in Principles of Plant Breeding, John Wiley and Son, pp. 115-161, 1960; Allard, 1960; Simmonds, 1979; Sneep et al., 1979; Fehr, 1987; Genetic Improvement of Vegetable Crops, Pergamon, pp. 197-234, Tatlioglu, 1993.

### Definitions

In the description that follows, a number of terms are used. In order to provide a clear and consistent understanding of the specification and claims, including the scope to be given such terms, the following definitions are provided:

Allele. The allele is any of one or more alternative forms of a gene, all of which relate to one trait or characteristic. In a diploid cell or organism, the two alleles of a given gene occupy corresponding loci on a pair of homologous chromosomes.

Backcrossing. Backcrossing is a process in which a breeder repeatedly crosses hybrid progeny back to one of the parents, for example, a first generation hybrid F₁ with one of the parental genotype of the F₁ hybrid.

Blossom end. The blossom end is the distal end of the fruit (the “far” end as measured from the base of the plant) where the flower blossom is located. The other end of a fruit is the stem end.

Covered cultivation. Any type of cultivation where the plants are not exposed to direct sunlight. The covering includes but is not limited to greenhouses, glasshouses, nethouses, plastic houses and tunnels.

Essentially all the physiological and morphological characteristics. A plant having essentially all the physiological and morphological characteristics means that the plants share essentially all physiological and morphological characteristics identified herein, except, e.g., where applicable, with respect to characteristics derived from a converted gene that differs between the plants as a result backcrossing, mutation, or genetic engineering.

Gene. As used herein, “gene” refers to a segment of nucleic acid. A gene can be introduced into a genome of a species, whether from a different species or from the same species, using transformation or various breeding methods.

Intermediate resistant or Intermediate resistance. Intermediate resistant refers to plant varieties that restrict the growth and development of the specified pest or pathogen, but may exhibit a greater range of symptoms or damage compared to highly resistant varieties. Intermediate resistant plant varieties will still show less severe symptoms or damage than susceptible plant varieties when grown under similar environmental conditions and/or pest or pathogen pressure.

Indeterminate Vine or Indeterminate Growth. Refers to apical meristem producing an unrestricted number of lateral organs; characteristic of vegetative apical meristems. (Anatomy of Seed Plants, 2nd Edition, 1977, John Wiley and Sons, page 513). The main stem of the plant continues to grow as long as the plant stays healthy, as opposed to a determinate plant, which at some point in its life cycle will stop growing longer.

Monoecious plant. A plant having staminate and pistillate flowers approximately equally present on the same plant.

Open habit. A plant with an “open habit” means a plant with small to medium sized leaves and reduced vigor of side shoots.

Parthenocarpic. “Parthenocarpic” refers to the ability of fruit to develop without pollination or fertilization. The fruit are therefore seedless when not pollinated, but can produce seeds if pollinated.

Propagate. To “propagate” a plant means to reproduce the plant by means including, but not limited to, seeds, cuttings, divisions, tissue culture, embryo culture or other in vitro method.

Quantitative Trait Loci (QTL). Quantitative trait loci refer to genetic loci that control to some degree numerically representable traits that are usually continuously distributed.

Regeneration. Regeneration refers to the development of a plant from tissue culture.

Single gene converted. Single gene converted or conversion plant refers to plants which are developed by a plant breeding technique called backcrossing wherein essentially all of the desired morphological and physiological characteristics of an inbred are recovered in addition to the single gene transferred into the inbred via the backcrossing technique, genetic engineering or mutation.

Vegetative propagation. Refers to taking part of a plant and allowing that plant part to form roots where plant part is defined as leaf, pollen, embryo, cotyledon, hypocotyl, meristematic cell, root, root tip, pistil, anther, flower, shoot tip, shoot, stem, fruit and petiole.

### Overview of the Hybrid ‘E97B.00040’

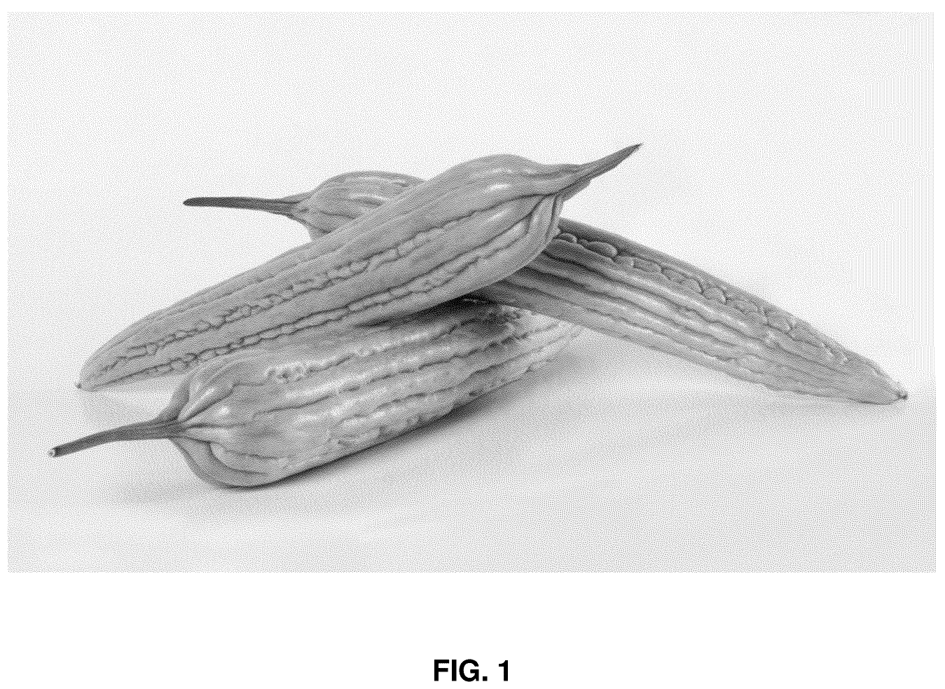

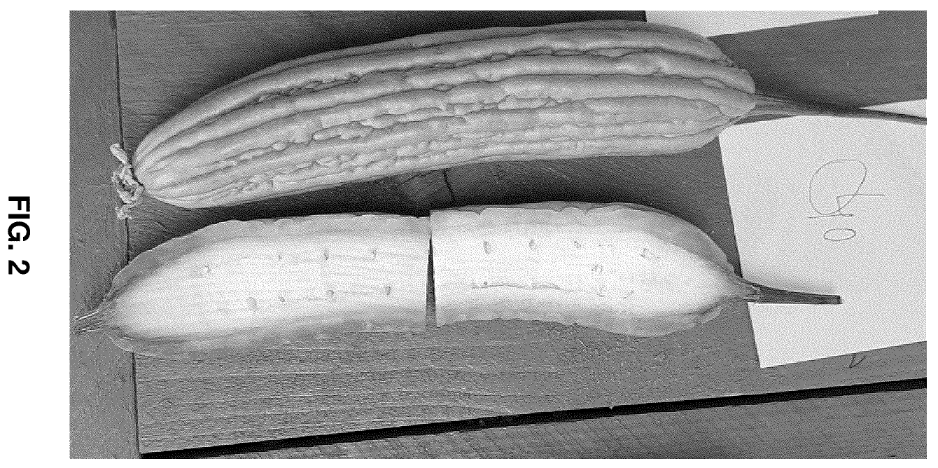

Hybrid bitter gourd ‘E97B.00040’ is a bitter gourd with medium green skin color at harvest maturity, few warts, long ridges, and oblong fruit in longitudinal section. Hybrid bitter gourd ‘E97B.00040’ is suitable for staked cultivation in greenhouses, particularly those excluding pollinating insects, in temperate areas, as well as hydroponic growing systems. FIGS. 1-2 depict fruits of hybrid bitter gourd ‘E97B.00040’. Hybrid bitter gourd ‘E97B.00040’ is the result of numerous generations of plant selections from its parent lines, and was chosen for its parthenocarpic fruit production without hormone treatment, fruit size (e.g., production of fruits having a weight of at least 200 grams), yield (e.g., continuous yield in the absence of pollinators), repellence of thrips and whitefly (e.g., due to its smell), and suitability for greenhouse production.

Hybrid bitter gourd ‘E97B.00040’ has shown uniformity and stability for the traits, within the limits of environmental influence for the traits. Hybrid bitter gourd ‘E97B.00040’ has been increased with continued observation for uniformity. No variant traits have been observed or are expected in ‘E97B.00040’.

### Objective Description of the Hybrid ‘E97B.00040’

In various embodiments, ‘E97B.00040’ can be characterized by the following morphologic and other characteristics as outlined below. The data which define these characteristics is based on observations taken in greenhouses located in Enkhuizen, Netherlands. Initial measurements of ‘E97B.00040’ were made 2-3 weeks after sowing, and after that plant and fruit measurements were made every 2 weeks.

Hybrid bitter gourd ‘E97B.00040’ has the following exemplary morphologic and other characteristics:

Main Usage: Fresh Market

Growing Condition:
Type of culture: Heated greenhouse, staked;
Suitable for hydroponic growing system: Yes
Special cultivation considerations: Production greenhouses should be free of
insects to produce parthenocarpic fruit; adapted to a single-stem, high-wire
production system
Growing region: Temperate areas
Planting period in adapted growth area: Year-round
Harvesting period in adapted growth area: Year-round
Mechanical harvest: Not adapted

Plant:
Number of nodes up to node with 1^{st} female flower: 5
Parthenocarpy: Present, without hormone treatment

Cotyledon:
Intensity of green color: Medium

Stem:
Length of internode of main stem (between 15^{th} and 20^{th} node): 12-15 cm
Thickness of main stem (between 15^{th} and 20^{th} node): 8-10 mm
Number of side shoots: Medium, when not pruned as a single stem

Leaf Blade:
Size: Medium
Intensity of green color: Medium
Ratio of lobe length to width: Medium
Number of lobes: Continuous range from five to seven
Depth of lobing: Medium

Petiole
Length: Medium

Ovary:
Length: 50 mm

Stigma:
Intensity of green color: Medium

Fruit:
Length: 25 to 27 cm
Diameter: 4 to 5 cm
Ratio of length to diameter: 6:1
Length of peduncle: 15 cm
Average weight: 220 grams
Maximum weight: 300 grams
Shape in longitudinal section: Oblong
Shape of base: Rounded
Shape of apex: Acute
Color of skin at market maturity: Medium green
Number of warts: Few
Wart size: Medium
Shape of wart top: Rounded
Presence of spines on wart: Absent
Length of ridge: Long
Color of skin at ripe stage: Orange
Bitterness: Present
Intensity of bitterness: Medium
Time of physiological maturity (when fruit is fully developed): Late

Seed:
Presence: Present when pollinated, absent when fruit set is parthenocarpic
Size: Medium
Number of seeds per fruit: 12-20
Intensity of brown color of testa: Medium
Indentation of seed edge: Medium

### Comparison of Hybrid Bitter Gourd ‘E97B.00040’ to Other Bitter Gourd Varieties

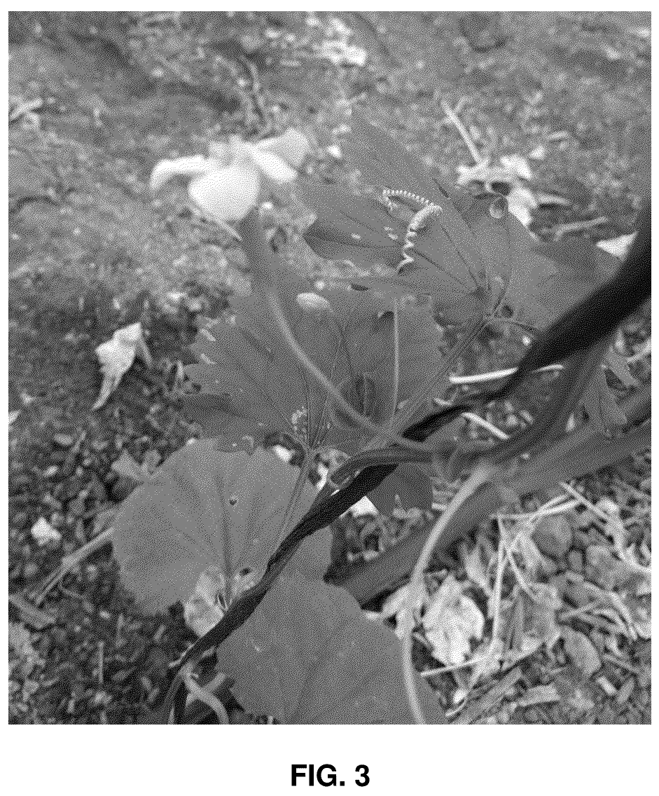

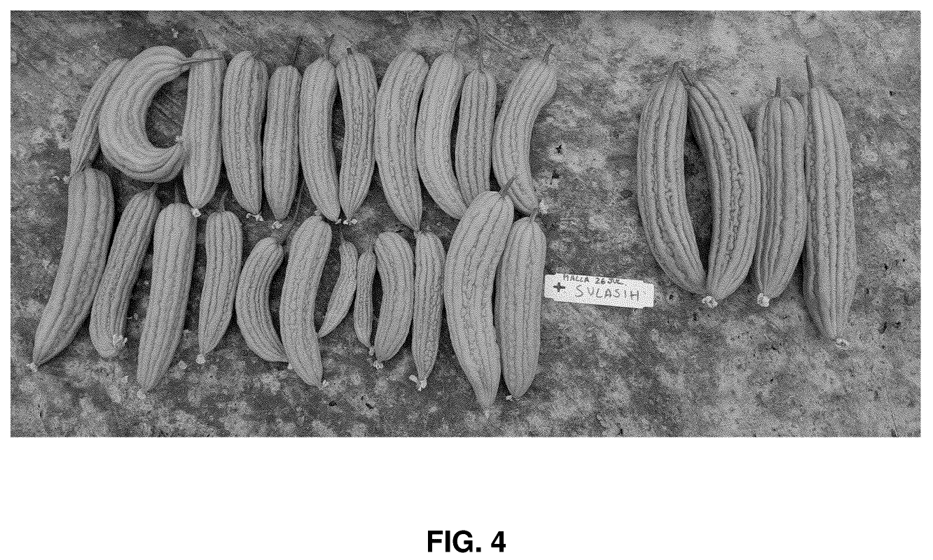

Hybrid bitter gourd ‘E97B.00040’ can be compared to bitter gourd variety ‘Bilai’. Column 1 of Table 1 shows the plant characteristics being compared, column 2 shows the characteristics of hybrid bitter gourd ‘E97B.00040’, and column 3 shows the characteristics of bitter gourd ‘Bilai’. Hybrid bitter gourd ‘E97B.00040’ is shown in FIGS. 1-3, and bitter gourd variety ‘Bilai’ is shown in FIG. 4.

TABLE 1
Comparison of hybrid bitter gourd ‘E97B.00040’ to ‘Bilai’.

| Characteristic | ‘E97B.00040’ | ‘Bilai’ | |
|---|---|---|---|
| Fruit length | 25-27 cm | 16-18 | cm |
| Fruit weight | 220 (avg)-300 (max) grams | 160-180 | grams |

| | | |
|---|---|---|
| Parthenocarpy | Parthenocarpic | Non-parthenocarpic |

### Further Embodiments

This disclosure also is directed to methods for producing a bitter gourd plant by crossing a first parent bitter gourd plant with a second parent bitter gourd plant wherein either the first or second parent bitter gourd plant is a hybrid bitter gourd plant of ‘E97B.00040’. Further, both first and second parent bitter gourd plants can come from the hybrid bitter gourd ‘E97B.00040’. All plants produced using hybrid bitter gourd ‘E97B.00040’ as a parent are within the scope of this disclosure, including plants derived from hybrid bitter gourd ‘E97B.00040’. Plants derived from hybrid bitter gourd ‘E97B.00040’ may be used, in certain embodiments, for the development of new bitter gourd varieties. By selecting plants having one or more desirable traits, a plant derived from hybrid bitter gourd ‘E97B.00040’ is obtained which possesses some of the desirable traits of the hybrid as well as potentially other selected traits.

The development of new varieties using one or more starting varieties is well known in the art. In accordance with this disclosure, novel varieties may be created by crossing hybrid bitter gourd ‘E97B.00040’ followed by multiple generations of breeding according to such well known methods. New varieties may be created by crossing with any second plant. In selecting such a second plant to cross for the purpose of developing novel lines, it may be desired to choose those plants which either themselves exhibit one or more selected desirable characteristics or which exhibit the desired characteristic(s) when in hybrid combination. Once initial crosses have been made, inbreeding and selection take place to produce new varieties. For development of a uniform line, often five or more generations of selfing and selection are involved.

It is preferred to breed for a combination of desirable plant characteristics and resistances to create a single variety or hybrid containing an improved combination of desirable traits from the parental germplasm. The development of commercial bitter gourd hybrids relates to the development and/or selection of bitter gourd parental lines, the crossing of these lines, and the evaluation of the crosses. Hybrid varieties offer multiple advantages, including a combination of desirable dominant and recessive traits from a set of inbred parents. Pedigree breeding and recurrent selection breeding methods are used to develop cultivars from breeding populations. Breeding programs combine desirable traits from two or more varieties or various broad-based sources into breeding pools from which lines are developed by selfing and selection of desired phenotypes. The new lines are crossed with other lines and the hybrids from these crosses are evaluated to determine which have the desirable characteristics.

As used herein, the term plant includes plant cells, plant protoplasts, plant cell tissue cultures from which bitter gourd plants can be regenerated, plant calli, plant clumps and plant cells that are intact in plants or parts of plants, such as embryos, pollen, ovules, flowers, leaves, stems, and the like.

### Genetic Marker Profile Through SSR and First Generation Progeny

In addition to phenotypic observations, the genotype of a plant can also be examined. There are many laboratory-based techniques known in the art that are available for the analysis, comparison and characterization of plant genotype. Such techniques include, without limitation, High Resolution Melting (HRM), DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites). For example, see Cregan et al., “An Integrated Genetic Linkage Map of the Soybean Genome” Crop Science 39:1464-1490 (1999), and Berry et al., “Assessing Probability of Ancestry Using Simple Sequence Repeat Profiles: Applications to Maize Inbred Lines and Soybean Varieties” Genetics 165:331-342 (2003).

Particular markers used for these purposes are not limited to any particular set of markers, but are envisioned to include any type of marker and marker profile, which provides a means of distinguishing varieties.

The present disclosure includes a hybrid bitter gourd plant characterized by molecular and physiological data obtained from the representative sample of said variety deposited with the National Collection of Industrial, Food and Marine Bacteria Ltd. (NCIMB Ltd.).

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by polymerase chain reaction (PCR), thereby eliminating the need for labor-intensive Southern hybridization. PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, include the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties it is preferable if all SSR profiles are performed in the same lab.

### Single-Gene Conversions

When the terms bitter gourd plant, cultivar, hybrid or bitter gourd line are used in the context of the present disclosure, this also includes any single gene conversions of that line. The term “single gene converted plant” as used herein refers to those bitter gourd plants which are developed by a plant breeding technique called backcrossing wherein essentially all of the desired morphological and physiological characteristics of a cultivar are recovered in addition to the single gene transferred into the line via the backcrossing technique. Backcrossing methods can be used with the present disclosure to improve or introduce a characteristic into the line. The term “backcrossing” as used herein refers to the repeated crossing of a hybrid progeny back to one of the parental bitter gourd plants for that line, backcrossing 1, 2, 3, 4, 5, 6, 7, 8 or more times to the recurrent parent. The parental bitter gourd plant which contributes the gene for the desired characteristic is termed the nonrecurrent or donor parent. This terminology refers to the fact that the nonrecurrent parent is used one time in the backcross protocol and therefore does not recur. The parental bitter gourd plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a bitter gourd plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original line. To accomplish this, a single gene of the recurrent cultivar is modified or substituted with the desired gene from the nonrecurrent parent, while retaining essentially all of the rest of the desired genetic, and therefore the desired physiological and morphological, constitution of the original line. The choice of the particular nonrecurrent parent will depend on the purpose of the backcross, one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new line but that can be improved by backcrossing techniques. Single gene traits may or may not be transgenic, examples of these traits include but are not limited to, male sterility, modified fatty acid metabolism, modified carbohydrate metabolism, herbicide resistance, resistance for bacterial, fungal, or viral disease, insect resistance, enhanced nutritional quality, industrial usage, yield stability and yield enhancement. These genes are generally inherited through the nucleus. Several of these single gene traits are described in U.S. Pat. Nos. 5,777,196, 5,948,957 and 5,969,212.

### Tissue Culture

Further reproduction of the variety can occur by tissue culture and regeneration. Tissue culture of various tissues of bitter gourd and regeneration of plants therefrom is well known and widely published. For example, reference may be had to Teng et al., HortScience. 1992, 27: 9,1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279,1441-1449, Nagata et al., Journal for the American Society for Horticultural Science. 2000,125: 6, 669-672, and Ibrahim et al., Plant Cell, Tissue and Organ Culture. (1992), 28(2): 139-145. It is clear from the literature that the state of the art is such that these methods of obtaining plants are routinely used and have a very high rate of success. Thus, another aspect of this disclosure is to provide cells which upon growth and differentiation produce bitter gourd plants having all, or essentially all, of the physiological and morphological characteristics of hybrid bitter gourd ‘E97B.00040’.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

Tissue culture of bitter gourd can be used for the in vitro regeneration of bitter gourd plants. Tissues cultures of various tissues of bitter gourd and regeneration of plants therefrom are well-known and published. By way of example, tissue cultures, some comprising organs to be used to produce regenerated plants, have been described in Burza et al., Plant Breeding. 1995,114: 4, 341-345, Cui Hongwen et al., Report Cucurbit Genetics Cooperative. 1999, 22, 5-7, Pellinen, Angewandte Botanik. 1997, 71: 3/4,116-118, Kuijpers et al., Plant Cell Tissue and Organ Culture. 1996, 46: 1, 81-83, Colijn-Hooymans et al., Plant Cell Tissue and Organ Culture. 1994, 39: 3, 211-217, Lou et al., HortScience. 1994, 29: 8, 906-909, Tabei et al., Breeding Science. 1994, 44: 1, 47-51, Sarmanto et al., Plant Cell Tissue and Organ Culture 31:3 185-193 (1992), Raharjo et al., Reports Cucurbits Genetics Cooperative 15, 35-39 (1992), Garcia-Sobo et al., Reports Cucurbits Genetics Cooperative 15, 40-44 (1992), Cade et al., Journal of the American Society for Horticultural Science 115:4 691-696 (1990), Chee et al., HortScience 25:7, 792-793 (1990), Kim et al., HortScience 24:4 702 (1989), and Punja et al., Plant Cell Report 9:2 61-64 (1990). It should also be mentioned that the regeneration of the bitter gourd after induction of adventitious shoot buds on calli derived from cotyledons has been described in Msikita et al., Cucurbit Genetics Cooperative Reports, 11: 5-7 (1988) and Kim et al., Plant Cell Tissue Organ Culture, 12: 67-74 (1988). Wehner et al., HortScience 16: 759-760 (1981) had previously described the induction of buds on cotyledons. Bitter gourd plants may be regenerated by somatic embryogenesis. These somatic embryos developed either in cell suspensions derived from calli developed from leaf explants (Chee et al., Plant Cell Report 7: 274-277 (1988)) or hypocotyls (Rajasekaran et al., Annals of Botany, 52: p. 417-420 (1983),) or directly on cotyledons (Cade et al., Cucurbit Genetics Cooperative Reports 11:3-4 (1988)) or leaf calli (Malepszy et al., Pflanzenphysiologie, 111: 273-276 (1983)). It is clear from the literature that the state of the art is such that these methods of obtaining plants are “conventional” in the sense that they are routinely used and have a very high rate of success. Thus, another aspect of this disclosure is to provide cells which upon growth and differentiation produce bitter gourd plants having all, or essentially all, of the physiological and morphological characteristics of hybrid bitter gourd ‘E97B.00040’.

### Additional Breeding Methods

The cultivar of the disclosure can also be used for transformation where exogenous genes are introduced and expressed by the cultivar of the disclosure. Genetic variants of ‘E97B.00040’ created either through traditional breeding methods or through transformation of hybrid bitter gourd ‘E97B.00040’ by any of a number of protocols known to those of skill in the art are intended to be within the scope of this disclosure.

Mutations for use in mutation breeding can be induced in plants by using mutagenic chemicals such as ethyl methane sulfonate (EMS), by irradiation of plant material with gamma rays or fast neutrons, or by other means. The resulting nucleotide changes are random, but in a large collection of mutagenized plants the mutations in a gene of interest can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). The principle of this method is based on the PCR amplification of the gene of interest from genomic DNA of a large collection of mutagenized plants in the M2 generation. By DNA sequencing or by looking for point mutations using a single-strand specific nuclease, such as the CEL-I nuclease (Till et al. (2004) Mismatch cleavage by single-strand specific nucleases. Nucleic Acids Res. 32, 2632-2641), the individual plants that have a mutation in the gene of interest are identified. By screening many plants, a large collection of mutant alleles is obtained, each giving a different effect on gene expression or enzyme activity. The gene expression or protein levels can for example be tested by transcript analysis levels (e.g., by RT-PCR) or by quantification of protein levels with antibodies. Plants with the desired reduced gene expression or reduced protein expression are then back-crossed or crossed to other breeding lines to transfer only the desired new allele into the background of the crop wanted.

Genes of interest for use in breeding may also be edited using gene editing techniques including transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques. For this, transgenic plants are generated expressing one or more constructs targeting the gene of interest. These constructs may include, without limitation, an anti-sense construct, an optimized small-RNA construct, an inverted repeat construct, a targeting construct, a guide RNA construct, a construct encoding a targeting protein, and/or a combined sense-anti-sense construct, and may work in conjunction with a nuclease, an endonuclease, and/or an enzyme, so as to downregulate the expression of a gene of interest.

One of ordinary skill in the art of plant breeding would know how to evaluate the traits of two plant varieties to determine if there is no significant difference between the two traits expressed by those varieties. For example, see Fehr and Walt, Principles of Cultivar Development, p. 261-286 (1987). Thus the disclosure includes hybrid bitter gourd ‘E97B.00040’ progeny bitter gourd plants comprising the ‘E97B.00040’ combination of traits listed in the Summary of the Disclosure, so that said progeny bitter gourd plant is not significantly different for said traits than bitter gourd ‘E97B.00040’ as determined at the 5% significance level when grown in the same environmental conditions and/or may be characterized by percent similarity or identity to hybrid bitter gourd ‘E97B.00040’ as determined by SSR markers. Using techniques described herein, molecular markers may be used to identify said progeny plant as a hybrid bitter gourd ‘E97B.00040’ progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

### Cultivation Methods

Advancements in bitter gourd cultivation are limited by niche demand for its fruit. In temperate areas, bitter gourd is typically grown on a small-scale as an annual open field crop, with production restricted to the warm season. Open field production of bitter gourd is similar to open field production of vining cucumber varieties, and typically relies on large trellises or straw mulches to prevent fruit damage from soil contact. Most commercial bitter gourd varieties require pollen vectors for fruit set, so yield may be poor in the absence of pollinating insects or hand pollination.

Because bitter gourd and vining cucumber have similar cultivation requirements, bitter gourd breeding programs may have similar breeding objectives to cucumber breeding programs. In particular, bitter gourd varieties suitable for heated greenhouses can extend fruit production in temperate areas. Traditional greenhouse cucumber production allows a main vine to grow vertically for a set distance before topping, which encourages hanging lateral shoots and formation of an umbrella-like habit. High-wire systems are a recent innovation in greenhouse cucumber production, and are a prospective new method for bitter gourd cultivation. High-wire systems train plants as a single growth point up a wire, and as plants grow, the heads are continuously lowered so fruit harvest remains at the same height. There are several potential benefits to high-wire systems: plant density can be much higher; fruit may be more uniform because no fruit are produced from lateral shoots; and pruning and harvest are simplified because the growing area is more predictably organized than traditional greenhouse cucumber production.

Certain traits are useful in high-wire systems. Favorable plant architecture includes short internodes, few lateral shoots, and small, horizontal leaves to promote an open habit. Disease resistance is another important consideration, as plants in a high-wire system must be kept healthy for longer than plants under conventional greenhouse conditions. An additional consideration for many cucurbits is parthenocarpy. Parthenocarpic varieties are useful for greenhouse production because fruit set does not rely on pollinating insects nor hand pollination; the complete exclusion of pollen vectors produces only seedless fruit, which can meet certain consumer preferences.

As used herein, the term “plant” includes plant cells, plant protoplasts, plant cell tissue cultures from which bitter gourd plants can be regenerated, plant calli, plant clumps and plant cells that are intact in plants or parts of plants, such as leaves, pollen, embryos, cotyledons, hypocotyl, roots, root tips, anthers, pistils, flowers, seeds, stems and the like.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### DEPOSIT INFORMATION

A deposit of at least 625 seeds of the bitter gourd variety ‘E97B.00040’ was made with the National Collection of Industrial, Food and Marine Bacteria Ltd (NCIMB Ltd), Wellheads Place, Dyce, Aberdeen, AB21 7 GB, United Kingdom, and assigned NCIMB Number X1. The seeds deposited with the NCIMB on DATE were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the bitter gourd variety ‘E97B.00040’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

1. A seed of hybrid bitter gourd designated as ‘E97B.00040’, representative sample of seed having been deposited under NCIMB Accession Number X1.

2. A bitter gourd plant produced by growing the seed of claim 1.

3. A plant part from the plant of claim 2.

4. The plant part of claim 3, wherein said part is a leaf, an ovule, a pollen grain, a fruit, or a cell.

5. The plant part of claim 4, wherein said part is a fruit.

6. A bitter gourd plant having all, or essentially all, of the physiological and morphological characteristics of the bitter gourd plant of claim 2.

7. A plant part from the plant of claim 6.

8. The plant part of claim 7, wherein said part is a leaf, an ovule, a pollen grain, a fruit, or a cell.

9. The plant part of claim 8, wherein said part is a fruit.

10. A pollen grain or an ovule of the plant of claim 2.

11. A protoplast produced from the plant of claim 2.

12. A tissue or cell culture of the plant of claim 2.

13. The tissue or cell culture of claim 12, wherein said tissue culture is produced from a plant part selected from the group consisting of root, root tip, meristematic cell, stem, hypocotyl, petiole, cotyledon, leaf, flower, anther, pollen, pistil, and fruit.

14. A bitter gourd plant regenerated from the tissue culture of claim 13, wherein the plant has all, or essentially all, of the morphological and physiological characteristics of a bitter gourd plant produced by growing hybrid bitter gourd seed designated as ‘E97B.00040’, representative sample of seed having been deposited under NCIMB Accession Number X1.

15. A method of producing a seed of a ‘E97B.00040’-derived bitter gourd plant, comprising:
(a) crossing a hybrid bitter gourd designated as ‘E97B.00040’, representative sample of seed having been deposited under NCIMB Accession Number X1, with itself or a second bitter gourd plant;
whereby seed of a ‘E97B.00040’-derived bitter gourd plant forms.

16. The method of claim 15, further comprising:
(b) crossing a plant grown from ‘E97B.00040’-derived bitter gourd seed with itself or a second bitter gourd plant to yield additional ‘E97B.00040’-derived bitter gourd seed;
(c) growing the additional ‘E97B.00040’-derived bitter gourd seed of step (b) to yield additional ‘E97B.00040’-derived bitter gourd plants; and
(d) repeating the crossing and growing of steps (b) and (c) for at least one additional generation to generate further ‘E97B.00040’-derived bitter gourd plants.

17. The method of claim 16, wherein the at least one additional generation comprises an additional 3-10 generations.

18. A method of vegetatively propagating a plant of hybrid bitter gourd variety ‘E97B.00040’, the method comprising the steps of:
(a) collecting tissue capable of being propagated from a plant of hybrid bitter gourd variety ‘E97B.00040’, representative seed of said hybrid bitter gourd variety having been deposited under NCIMB Accession Number X1; and
(b) producing a rooted plant from said tissue.

19. A method of producing a bitter gourd fruit, said method comprising growing the plant of claim 2 until it sets at least one fruit, and harvesting the fruit.
